(19) United States
(12) Patent Application Publication
(10) Pub. No.: US 2004/0162607 A1
(43) Pub. Date: Aug. 19, 2004

(54) Prosthetic arterial graft with test port

(76) Inventor: Saqib Masroor, Greenville, NC (US)

Correspondence Address: Mark Terry, 227 Michigan Av. #204, Miami Beach, FL, 33139, US

(21) Appl. No.: 10/748,635

(22) Filed: Dec. 30, 2003

Related U.S. Application Data
(60) Provisional application No. 60/436,924, filed on Dec. 30, 2002.

(51) Int. Cl. A61F 2/06

(52) U.S. Cl. 623/1.24; 606/154

(57) ABSTRACT

A method and apparatus for performing prosthetic arterial graft surgery is disclosed. The apparatus for facilitating prosthetic arterial graft surgery includes a tubular element having a first end and a second end exposing the inside volume of the tubular element, the first end and the second end for anastomotic coupling to an artery. The apparatus further includes an enclosure coupled to a side of the tubular element, wherein an inside volume of the enclosure is continuous with the inside volume of the tubular element and an access valve coupled to the enclosure, allowing for insertion of fluid into the enclosure. It is determined whether the prosthetic arterial graft is hemostatic by insertion of fluid into the tubular element via the access valve and inspecting for leaking of the fluid at the anastomoses.

### CROSS-REFERENCED APPLICATIONS

This U.S. patent application claims priority to U.S. Provisional Patent Application No. 60/436,924, entitled “Prosthetic Arterial Graft with Test Port” and filed by Saqib Masroor on Dec. 30, 2002. The aforementioned U.S. Provisional Patent Application is hereby incorporated by reference in its entirety.

### PARTIAL WAIVER OF COPYRIGHT

All of the material in this patent application is subject to copyright protection under the copyright laws of the United States and of other countries. As of the first effective filing date of the present application, this material is protected as unpublished material. However, permission to copy this material is hereby granted to the extent that the copyright owner has no objection to the facsimile reproduction by anyone of the patent documentation or patent disclosure, as it appears in the United States Patent and Trademark Office patent file or records, but otherwise reserves all copyright rights whatsoever.

### FIELD OF THE INVENTION

This invention generally relates to the field of prosthetic arterial grafting and more specifically to anastomoses testing during prosthetic arterial grafting.

### DESCRIPTION OF RELATED ART

Replacement of native arteries with prosthetic grafts is a commonly performed procedure today, for a variety of different indications in many different anatomical regions of the human body. This includes repair of aortic aneurysms, less common non-aortic aneurysms, aortoiliac occlusive disease, upper and lower extremity revascularizations, brachiocephalic and other arch vessel reconstruction and mesenteric revascularizations.

The incidence of abdominal aortic aneurysms has been reported to be 21 per 100,000 person-years (Bickerstaff L K, Hollier L H, Van Peenen H J, et al: Abdominal aortic aneurysms: The changing natural history. J Vasc Surg 1984;1:6) and that of thoracic aneurysms up to 10.4 per 100,000 person-years (Clouse W D, Hallett J W Jr., Schaff H V, et al. Improved prognosis of thoracic aortic aneurysms: a population based study. JAMA 280 (22): 1926-9,1998). Abdominal aortic aneurysms (AAA) are associated with the highest risk of rupture of up to 50% per year once they reach a size of 8 cm, a condition that carries a mortality of 50% in people who make it to the hospital. It should be noted that half of the people with ruptured abdominal aneurysms die before reaching the hospital. Ruptured AAA is the 15th leading cause of death in men in the United States (Cronenwett J L, Krupski W C and Rutherford R B. Abdominal aortic and iliac aneurysms).

To prevent this catastrophic event, prophylactic repair of abdominal aortic aneurysms is performed once they reach a critical diameter. In the United States alone, 40,000 AAA repairs are performed annually and this procedure is one of the most common vascular surgery procedures performed (Gillum R F. Epidemiology of aortic aneurysm in the United States. J Clin Epidemiol 1995; 48:1289). The conventional treatment involves replacement of the aneurysmal (dilated) aorta with a prosthetic graft. In textile or fabric grafts such as Dacron grafts, the basic polymer is made into a yarn, which is then knit or woven into a graft (Brewster D C. Prosthetic Grafts. In Rutherford (Ed) Vascular Surgery. 5th Edition. W. B. Saunders Company, Philadelphia, 2000). In case of non-textile grafts such as expanded polytetrafluoroehtylene (ePTFE) grafts, the technique of precipitation of the polymer from solution of the material is used for construction of the graft (see Brewster D C). The grafts are either straight (for aorti-iliac anastomosis) or bifurcated (for aorto-bifemoral anastomosis).

The surgical procedure for repair of AAA involves clamping the aorta proximally and distally, thus halting the blood flow across the aneurysmal aortic segment, followed by suturing of the prosthetic graft in place of the diseased aorta. At the completion of these anastomoses, the clamps are removed, thus restoring blood flow through the interposed prosthetic graft. One of the most common complications of this type of surgery is hemorrhage. Hemorrhage most commonly occurs from the proximal aortic anastomosis or from iatrogenic venous injury. This is even more common in thoracic and thoracoabdominal aortic aneurysms requiring cardiopulmonary bypass with or without circulatory arrest. Hemorrhage is not just a cause of increased morbidity but also increased mortality. Presently, the only way of finding out whether an anastomosis is hemostatic (i.e., having no leaks) is to release the clamp and restore the blood flow across the anastomosis. Once a bleeding point is localized and found to be easily approachable, a surgeon can attempt to control it with more hemostatic stitches. Not uncommonly, the bleeding site is inaccessible and the patient's aorta then needs to be re-clamped.

Alternatively, sometimes the patient is required to return to cardiopulmonary bypass in preparation for re-clamping. These attempts at controlling bleeding are not without their drawbacks. Potential clamp injury can occur to the already friable aorta when it is clamped the second time or more. Also, additional clamping can cause injury to adjacent organs, such as the inferior vena cava and renal artery and vein (in case of AAA), pulmonary artery, left subclavian artery, left carotid artery and esophagus (in case of thoracic aneurysms). Further, prolonged clamping can lead to worsening coagulopathy.

The best way to treat this potentially lethal complication is to prevent it. In severely atherosclerotic arteries it is even more important that there is minimal handling of the arteries. Thus, it is desirable to have a single clamp rather than multiple clamp applications. Also, bleeding can be better controlled if the stitches are placed in a more controlled environment where there is good exposure of the leaks without blood obscuring the vision. At present there is no way of testing the hemostasis of the newly constructed anastomosis besides removal of clamps with resumption of blood flow across the graft. The bleeding sites are then controlled with more stitches while the anastomosis is bleeding. If this is not possible because of excessive bleeding obscuring the field of view, the aorta has to be re-clamped and the entire anastomosis has to be redone. Potential bleeding sites can be fixed much more easily and in a more controlled fashion if done before resumption of blood flow and release of clamps. It is therefore important to devise a means of testing the hemostatic nature of the anastomosis in order to prevent the potentially fatal and common complication of anastomotic bleeding and adjacent organ injury.

Another complication of vascular reconstruction surgery with a prosthetic graft relates to embolism of intravascular debris to distal tissues. This occurs when clamps are first placed on the artery as well as when they are removed. At these stages, calcified fractured atherosclerotic plaques and debris get dislodged and flow downstream to block blood flow to distal organs such as lower extremities (in case of AAA), brain (causing stroke in ascending aortic replacement) etc. This is currently prevented by releasing the clamps one at a time before tying down the last couple of stitches of the second anastomosis and allowing the anastomosis to bleed. This allows the egress of any debris or air out of the vascular system through the anastomosis. Once this venting has been accomplished, the stitch is tied down to complete the anastomosis and both the clamps are removed to resume blood flow to the distal areas. As can be imagined, this is a very uncontrolled maneuver with potential for more hemorrhage.

Therefore a need exists to overcome the problems with the prior art as discussed above, and particularly for a way to perform prosthetic arterial grafting more efficiently.

### SUMMARY OF THE INVENTION

The present invention, according to a preferred embodiment, overcomes problems with the prior art by providing an efficient and easy-to-implement apparatus for performing a prosthetic arterial graft surgery.

A method and apparatus for performing prosthetic arterial graft surgery is disclosed. In an embodiment of the present invention, the apparatus for facilitating prosthetic arterial graft surgery includes a tubular element having a first end and a second end exposing the inside volume of the tubular element, the first end and the second end for anastomotic coupling to an artery. The apparatus further includes an enclosure coupled to a side of the tubular element, wherein an inside volume of the enclosure is continuous with the inside volume of the tubular element and an access valve coupled to the enclosure, allowing for insertion of fluid into the enclosure. It is determined whether the prosthetic arterial graft is hemostatic by insertion of fluid into the tubular element via the access valve and inspecting for leaking of the fluid at the anastomoses.

Further disclosed is a method for performing prosthetic arterial graft surgery. The method includes creating a first opening of an artery and a second opening of the artery and clamping the first opening of the artery and the second opening of the artery to prevent bleeding. The method further includes surgically coupling a prosthetic graft to the first opening of the artery and the second opening of the artery, wherein the prosthetic graft comprises a tubular element having a first end and a second end exposing the inside volume of the tubular element, the first end surgically coupled to the first opening and the second end surgically coupled to the second opening. The prosthetic graft further includes an enclosure coupled to a side of the tubular element, wherein an inside volume of the enclosure is continuous with the inside volume of the tubular element and an access valve coupled to the enclosure, allowing for insertion of fluid into the enclosure. The method further includes inserting fluid into the tubular element via the access valve and inspecting for leaking of the fluid at the coupling of the first opening and the first end and the coupling of the second opening and the second end.

In an embodiment of the present invention, the method further includes surgically repairing the coupling that is leaking if leaking is detected upon inspection. The method further includes unclamping any one of the first opening and the second opening to allow flow of blood through the prosthetic graft and allowing egress of debris from the access valve. The method further includes unclamping any one of the first opening and the second opening to allow flow of blood through the prosthetic graft and allowing egress of air from the access valve.

The foregoing and other features and advantages of the present invention will be apparent from the following more particular description of the preferred embodiments of the invention, as illustrated in the accompanying drawings.

### BRIEF DESCRIPTION OF THE DRAWINGS

The subject matter, which is regarded as the invention, is particularly pointed out and distinctly claimed in the claims at the conclusion of the specification. The foregoing and other features and also the advantages of the invention will be apparent from the following detailed description taken in conjunction with the accompanying drawings.

FIG. 1 is an illustration of a side view of a prosthetic graft of one embodiment of the present invention.

FIG. 2 is an illustration of a side view of the prosthetic graft of FIG. 1.

FIG. 3 is an illustration of a side view of the prosthetic graft of FIG. 1 in use during a prosthetic graft surgery.

FIG. 4 is an illustration of a side view of a prosthetic graft with a port, in one embodiment of the present invention.

FIG. 5 is an illustration of a side view of a prosthetic graft with a port and connector, in one embodiment of the present invention.

### DETAILED DESCRIPTION OF THE PREFERRED EMBODIMENTS

It should be understood that these embodiments are only examples of the many advantageous uses of the innovative teachings herein. In general, statements made in the specification of the present application do not necessarily limit any of the various claimed inventions. Moreover, some statements may apply to some inventive features but not to others. In general, unless otherwise indicated, singular elements may be in the plural and vice versa with no loss of generality. In the drawing like numerals refer to like parts through several views.

The prosthetic graft of the present invention includes a valve or side port coming off of the main body of the graft. Attached to this side port may be a reservoir of a pressurized liquid, such as 0.9% Sodium Chloride solution, which can be released into the graft when needed. This solution, injected into the graft under pressure, can demonstrate the potential bleeding sites at both the proximal and distal anastomosis, which can then be adequately controlled by the surgeon prior to removal of the surgical clamp. The entire operation can thus be performed under one clamp in a controlled environment, without needlessly injuring the artery and adjacent organs by repeated, potentially blinded applications of a clamp (when anastomotic bleeding is encountered).

Once hemostasis has been assured, the valve serves another important function, and that is de-airing and egress of intravascular debris in these diseased blood vessels. Once both these functions have been performed, the valve can be simply clipped and ligated flush with the wall of the prosthetic graft. This device can be used with grafts of any caliber, length, shape or design (tube or bifurcated).

FIG. 1 is an illustration of a side view of a prosthetic graft 100 of one embodiment of the present invention. FIG. 1 shows a tubular element measuring 10-50 cm in length comprising the prosthetic graft 100. FIG. 1 further shows one opening on each end of the prosthetic graft 100-an opening 102 and an opening 104. Each opening 102, 104 is surgically coupled (such as through suturing) with a tubular vascular element such as a vein, an artery, any other type of blood vessel or a prosthetic graft. This is described in greater detail below. Thus, the diameter of the prosthetic graft 100 must accommodate the diameter the vascular element to which the prosthetic graft 100 is coupled, such that the prosthetic graft 100 fits securely within the vascular element. That is, the diameter of the prosthetic graft 100 is substantially the same as the diameter of the vascular element to which the prosthetic graft 100 is coupled. Thus, the diameter of the prosthetic graft 100 is from about 4 mm to about 30 mm, depending on the vascular element to which the prosthetic graft 100 is coupled.

FIG. 1 further shows that the lumen 120 of the prosthetic graft 100 must be substantially wide to allow a proper amount of blood to flow through the prosthetic graft 100. For this reason, the inside diameter of the prosthetic graft 100 can be from about 6 mm to about 36 mm.

FIG. 1 further shows a valve or cap 108 that is attached to the tubular element of the prosthetic graft 100. The valve 108 is hingably coupled to the tubular element of the prosthetic graft 100 via a hinge 106.

In an embodiment of the present invention, the prosthetic graft 100 is composed of any material suitable for surgical coupling With an artery. It should be noted that although FIG. 1 shows prosthetic graft 100 to be formed in a simple cylindrical shape, the present invention supports any number of shapes for prosthetic graft 100, such as a tube shape (such as used in ascending or descending thoracic aorta and less commonly in AAA) or a bifurcated shape (such as in AAA requiring aortobifemoral anastomoses).

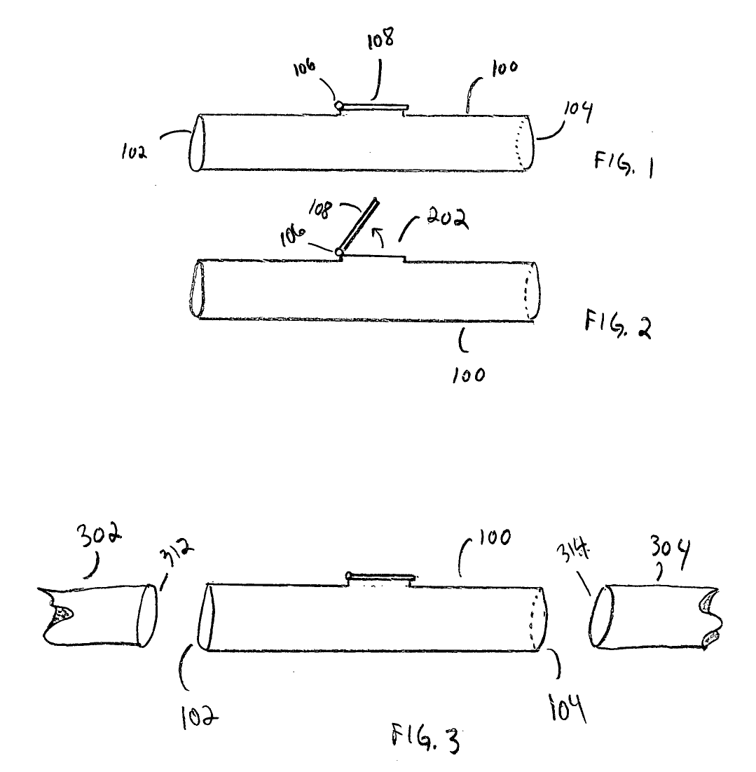

FIG. 2 is an illustration of a side view of the prosthetic graft 100 of FIG. 1. FIG. 2 shows the prosthetic graft 100 with the valve 108 opened via the hinge 106. This allows the volume within the tubular element of prosthetic graft 100 to be exposed. In this way, liquids, solids or gas can be extracted from the volume of the tubular element of prosthetic graft 100. This is described in greater detail below. Moreover, liquids, solids or gas can be inserted into or introduced for ingress into the volume of the tubular element of prosthetic graft 100 via the valve 108. In one embodiment of the present invention, when the valve 108 is closed, liquid may be inserted into the tubular element of the prosthetic graft 100 by inserting a syringe or other piercing object into the valve 108 and introducing liquid into the prosthetic graft 100.

It should be noted that although FIGS. 1-2 shows only one type of valve 108, the present invention supports any type of valve for prosthetic graft 100, such as a spigot, a stopcock, and a pressure or squeeze valve, which allows the ingress or egress of liquids, solids or gases into the inner volume of the prosthetic graft 100.

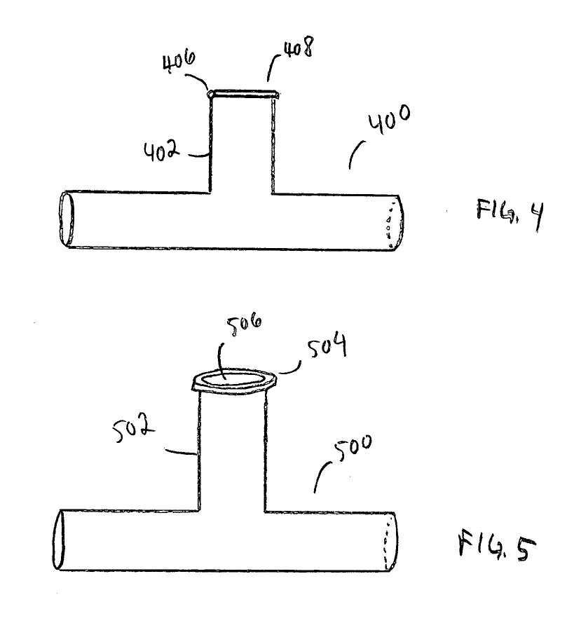

FIG. 3 is an illustration of a side view of the prosthetic graft 100 of FIG. 1 in use during a prosthetic graft surgery. FIG. 3 shows the prosthetic graft 100 positioned between two vascular elements—vascular element 302 and vascular element 304. The opening 102 of the prosthetic graft 100 shall be surgically coupled with the end 312 of the vascular element 302 and the opening 104 of the prosthetic graft 100 shall be surgically coupled with the end 314 of the vascular element 304. A prosthetic graft surgery will now be described.

The prosthetic graft 100 of the present invention can be cut to the required size keeping the valve 108 in the center of the operative field. It should be assured that when beginning the anastomosis, the valve 108 comes off at the top of the circumference of the prosthetic graft 100 and not from its sides. This is because the valve 108 can also be used for de-airing and so it is important that it is the highest point of the prosthetic graft 100. Either the proximal (vascular element 302) or distal (vascular element 304) anastomosis can be constructed first. At the completion of this anastomosis the other end of the prosthetic graft 100 can be clamped and the prosthetic graft 100 filled with liquid through the valve 108 under pressure.

The use of stiff inelastic tubing down to the proximal 5 mm of the valve 108 allows more accurate transmission of pressure within the prosthetic graft 100, while still allowing the surgeon to close the valve 108 easily at the end of the procedure at the base of the valve 108 (where the valve 108 meets the tubular element of the prosthetic graft 100). This maneuver will demonstrate any leaks in the anastomosis that can then be fixed. Alternatively, the whole anastomosis can be redone, without any additional handling of the diseased artery or vascular elements. Once satisfied of the adequacy of this anastomosis, the other anastomosis can be constructed and again, without releasing arterial clamps, the prosthetic graft 100 can be filled with 0.9% NaCI solution from the valve 108 under pressure to test the anastomosis. The prosthetic graft 100 might be tested once only at the completion of both the proximal and distal anastomoses.

Once the surgeon is satisfied with both the anastomoses, the valve 108 is opened to air. It can then be used to vent the prosthetic graft 100 and flush any intravascular debris that may have accumulated at the proximal and distal clamps. The prosthetic graft 100 is de-aired sequentially by release of first the distal and then the proximal clamp, while allowing the prosthetic graft 100 to bleed through the valve 108. Once satisfied that it has been de-aired and free of any distal or proximal debris, the valve 108 can be clipped and suture-ligated at the base of the valve 108, where the valve 108 meets the tubular element of the prosthetic graft and which can be made of the graft material itself.

In one embodiment of the present invention, the present invention can be used during aortic valve-sparing operations to test the viability of aortic valves. In aortic valve-sparing operations on the aortic root using a prosthetic graft 100 of the construction of the present invention will allow the surgeon to test the competency of the patient's native aortic valve attached to the prosthetic graft. In this embodiment, an anastomosis is performed, connecting each opening of the prosthetic graft 100 to the aorta near the aortic valve. Subsequently, the prosthetic graft 100 is placed under pressure by inserting liquid or other substance into the inner volume of the prosthetic graft 100. If the aortic valve is functioning properly, the pressure within the inner volume of the prosthetic graft 100 will be maintained and will not decrease. This is because the aortic valve only allows blood to flow in one direction. If the aortic valve is not functioning properly, the pressure within the inner volume of the prosthetic graft 100 will decrease as the fluid within the graft flows into the heart, which will become distended. In this way, the viability of an aortic valve is tested.

The features of the present invention are advantageous as they reduce the number of times vascular elements must be clamped during the prosthetic graft surgery, thereby reducing complications and other negative implications. The present invention also reduces the time required for surgery and thereby lowers the risks of infections and other complications. The present invention further allows for the de-airing a removal of debris from the inner volume of the graft, which eliminates the risk of embolism or other side effects associated with the release of a foreign object into the blood stream.

Further, the features of the present invention are advantageous as bleeding can be better controlled since there is good exposure of the leaks at the anastomosis without blood obscuring the vision. Potential bleeding sites can be fixed much more easily and in a more controlled fashion since fixing of leaks is performed before resumption of blood flow and release of clamps. The present invention is advantageous because it provides a means of testing the hemostatic nature of the anastomosis in order to prevent the potentially fatal and common complication of anastomotic bleeding and adjacent organ injury. The present invention is further advantageous because it provides a means for testing an aortic valve while reducing the number of clamps required as well as reducing the time required for the surgery, thereby reducing surgical complications.

FIG. 4 is an illustration of a side view of a prosthetic graft 400 with a port 402, in one embodiment of the present invention. FIG. 4 shows a prosthetic graft 400 similar in size, construction and composition as prosthetic graft 100 of FIG. 1. FIG. 4 further shows a port 402, which is an enclosure of substantially cylindrical shape that is coupled to the prosthetic graft 400 perpendicularly. The port 402 includes a cylindrical hollow volume that is continuous with the cylindrical hollow volume of the tubular element of the prosthetic graft 400.

The port 402 can consist of a 10 cm long tubular element of 3-5 mm diameter. The port 402 should be such that it can be easily bled into a container away from the anastomosis when the prosthetic graft 400 is being de-aired, rather than bleeding into the operative field. The proximal 5 mm of the port 402 attached to the body of the prosthetic graft 400 can be constructed of the same material as the rest of the prosthetic graft 400. The distal 9.5 cm of the port 402 can be made of non-elastic plastic tubing (such as ones used for transducing blood pressure)

FIG. 4 further shows a valve or cap 408 that is attached to the tubular element of the prosthetic graft 400. The valve 408 is hingably coupled to the tubular element of the prosthetic graft 400 via a hinge 406.

FIG. 5 is an illustration of a side view of a prosthetic graft 500 with a port 502 and connector 504, in one embodiment of the present invention. FIG. 5 shows a prosthetic graft 500 similar in size, construction and composition as prosthetic graft 100 of FIG. 1. FIG. 5 further shows a port 502, which is an enclosure of substantially cylindrical shape that is coupled to the prosthetic graft 500 perpendicularly. The port 502 includes a cylindrical hollow volume that is continuous with the cylindrical hollow volume of the tubular element of the prosthetic graft 500.

The prosthetic graft 500 also includes a connector 506, which can be a conventional male-female connector used in clinical practice for intravenous and arterial pressure monitoring lines. For purposes of filling the prosthetic graft 500 with fluid (see description above of prosthetic graft surgery), a sterile arterial line tubing can be connected to the connector 506 at one end and the other end will be passed off the operating table to a pressurized liquid reservoir such as a bag of 0.9% NaCI solution in a pressure bag. This bag can be pressurized to the desired pressure of 250-300 mm Hg. Normal systolic blood pressure in humans is 120, however pressures of 200 mmHg or even higher are often encountered in hypertensive patients especially postoperatively.

It should be noted that although FIG. 5 shows one type of connector 506 for the prosthetic graft 500, the present invention supports any type of connector 506 for prosthetic graft 100, such as a screw connector, a fitted valve connector or a pressure connector.

### CONCLUSION

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments. Furthermore, it is intended that the appended claims cover any and all such applications, modifications, and embodiments within the scope of the present invention.

What is claimed is:

1. A prosthetic arterial graft apparatus, comprising:
a tubular element having a first end and a second end exposing the inside volume of the tubular element, the first end and the second end for surgical coupling to an artery; and
an access valve coupled to the tubular element, allowing for insertion of fluid into the inside volume of the tubular element;
wherein it is determined whether the prosthetic arterial graft is hemostatic by insertion of fluid into the tubular element via the access valve and inspecting for leaking of the fluid at the coupling between the first end and the artery and the coupling between the second end and the artery.

2. The apparatus of claim 1, wherein the access valve allows for egress of debris from the inside volume of the tubular element.

3. The apparatus of claim 2, wherein the access valve allows for egress of gas from the inside volume of the tubular element.

4. The apparatus of claim 1, wherein the tubular element is a cylindrical shaped element constructed of any one of polytetrafluoroehtylene and Dacron.

5. The apparatus of claim 1, further comprising a connector disposed on the access valve for coupling to a device for insertion of fluid into the tubular element.

6. A prosthetic arterial graft apparatus, comprising:
a tubular element having a first end and a second end exposing the inside volume of the tubular element, the first end and the second end for anastomotic coupling to an artery;
an enclosure coupled to a side of the tubular element, wherein an inside volume of the enclosure is continuous with the inside volume of the tubular element; and
an access valve coupled to the enclosure, allowing for insertion of fluid into the enclosure;
wherein it is determined whether the prosthetic arterial graft is hemostatic by insertion of fluid into the tubular element via the access valve and inspecting for leaking of the fluid at the anastomoses.

7. The apparatus of claim 6, wherein the access valve allows for egress of debris from the inside volume of the tubular element.

8. The apparatus of claim 7, wherein the access valve allows for egress of gas from the inside volume of the tubular element.

9. The apparatus of claim 6, wherein the tubular element is a cylindrical shaped element constructed of any one of polytetrafluoroehtylene and Dacron.

10. The apparatus of claim 6, wherein the enclosure is a cylindrical shaped element perpendicularly disposed on a midsection of the side of the tubular element.

11. The apparatus of claim 6, further comprising a connector disposed on the access valve for coupling to a device for insertion of fluid into the tubular element.

12. A method of performing a prosthetic arterial graft surgery, comprising:
creating a first opening of an artery and a second opening of the artery;
clamping the first opening of the artery and the second opening of the artery to prevent bleeding;
surgically coupling a prosthetic graft to the first opening of the artery and the second opening of the artery, wherein the prosthetic graft comprises:
a tubular element having a first end and a second end exposing the inside volume of the tubular element, the first end surgically coupled to the first opening and the second end surgically coupled to the second opening;
an enclosure coupled to a side of the tubular element, wherein an inside volume of the enclosure is continuous with the inside volume of the tubular element; and
an access valve coupled to the enclosure, allowing for insertion of fluid into the enclosure;
inserting fluid into the tubular element via the access valve and inspecting for leaking of the fluid at the coupling of the first opening and the first end and the coupling of the second opening and the second end.

13. The method of claim 12, further comprising:
if leaking is detected upon inspection, surgically repairing the coupling that is leaking.

14. The method of claim 13, further comprising:
unclamping any one of the first opening and the second opening to allow flow of blood through the prosthetic graft; and
allowing egress of debris from the access valve.

15. The method of claim 14, further comprising:
unclamping any one of the first opening and the second opening to allow flow of blood through the prosthetic graft; and
allowing egress of air from the access valve.

16. The method of claim 12, wherein the step of creating a first opening of an artery and a second opening of the artery comprises any one of:
severing and bypassing the artery;
severing and excising a portion of the artery; and
excluding the artery.
